(19) United States
(12) Patent Application Publication
(10) Pub. No.: US 2025/0195021 A1
(43) Pub. Date: Jun. 19, 2025

(54) MEDICAL IMAGE DISPLAY APPARATUS, MEDICAL IMAGE DISPLAY SYSTEM, STORAGE MEDIUM, AND MEDICAL IMAGE DISPLAY METHOD

(71) Applicant: Konica Minolta, Inc., Tokyo (JP)

(72) Inventor: Tomoaki TAKEMURA, Tokyo (JP)

(21) Appl. No.: 18/975,336

(22) Filed: Dec. 10, 2024

(30) Foreign Application Priority Data
Dec. 13, 2023 (JP) 2023-210229

(51) Int. Cl. A61B 6/46; A61B 6/00; G06T 7/00

(52) U.S. Cl. CPC A61B 6/463; A61B 6/5217; G06T 7/0016; G06T 2207/10081; G06T 2207/10088; G06T 2207/30096

(57) ABSTRACT

A medical image display apparatus comprising a hardware processor that acquires a series of medical images including a plurality of medical images, performs image analysis on the plurality of medical images to extract a plurality of medical images each including a lesion candidate region, for at least two medical images among the extracted plurality of medical images each including the lesion candidate region, calculates lesion information on the lesion candidate region included in each of the at least two medical images, and based on the lesion information on each of the at least two medical images from each of which the lesion information has been calculated, controls display of information indicating presence of the lesion candidate region about each of the plurality of medical images each including the lesion candidate region.

### BACKGROUND OF THE INVENTION

### Technical Field

The present invention relates to a medical image display apparatus, a medical image display system, a storage medium, and a medical image display method.

### Description of Related Art

Conventionally, a technique is known in which a series of medical images including a plurality of medical images is subjected to image analysis and the position of a medical image including a lesion candidate region is displayed on a slider bar. For example, in Japanese Unexamined Patent Publication No. 2004-173910, there is disclosed displaying, on a slider bar, a mark indicating the position of a sectional image in which an abnormal shadow candidate has been detected, among a plurality of sectional images constituting a CT image. In Japanese Unexamined Patent Publication No. 2004-173910, there is also disclosed, when displaying the above-described mark, performing display so as to distinguish a display-done object and a dimply-not-done object from one another.

### SUMMARY OF THE INVENTION

Incidentally, in a case where medical images are analyzed by computer aided diagnosis (CAD), artificial intelligence (AI), or the like to detect lesion candidate regions, the number of detected lesion candidate regions has increased due to improvement in detection performance. Therefore, in the technology disclosed in Japanese Unexamined Patent Publication No. 2004-173910, the number of marks indicating the positions of sectional images including lesion candidate regions to be displayed on the slider bar increases excessively, which causes a problem that it cannot understand which sectional image should be preferentially viewed.

The present invention has been made in consideration of the above problems, and an object thereof is to display, in an easily understandable manner, which medical image should be preferentially viewed among a plurality of medical images each including a lesion candidate region.

To achieve at least one of the abovementioned objects, according to an aspect of the present invention, a medical image display apparatus reflecting one aspect of the present invention includes a hardware processor that
acquires a series of medical images including a plurality of medical images,
performs image analysis on the plurality of medical images to extract a plurality of medical images each including a lesion candidate region,
for at least two medical images among the extracted plurality of medical images each including the lesion candidate region, calculates lesion information on the lesion candidate region included in each of the at least two medical images, and
based on the lesion information on each of the at least two medical images from each of which the lesion information has been calculated, controls display of information indicating presence of the lesion candidate region about each of the plurality of medical images each including the lesion candidate region.

According to an aspect of the present invention, a medical image display system reflecting one aspect of the present invention includes a hardware processor that
acquires a series of medical images including a plurality of medical images,
performs image analysis on the plurality of medical images to extract a plurality of medical images each including a lesion candidate region,
for at least two medical images among the extracted plurality of medical images each including the lesion candidate region, calculates lesion information on the lesion candidate region included in each of the at least two medical images, and
based on the lesion information on each of the at least two medical images from each of which the lesion information has been calculated, controls display of information indicating presence of the lesion candidate region about each of the plurality of medical images each including the lesion candidate region.

According to an aspect of the present invention, a non-transitory computer-readable storage medium reflecting one aspect of the present invention stores a program causing a computer to perform:
acquiring a series of medical images including a plurality of medical images;
performing image analysis on the plurality of medical images to extract a plurality of medical images each including a lesion candidate region;
for at least two medical images among the extracted plurality of medical images each including the lesion candidate region, calculating lesion information on the lesion candidate region included in each of the at least two medical images; and
based on the lesion information on each of the at least two medical images from each of which the lesion information has been calculated, controlling display of information indicating presence of the lesion candidate region about each of the plurality of medical images each including the lesion candidate region.

According to an aspect of the present invention, a medical image display method reflecting one aspect of the present invention includes:
acquiring a series of medical images including a plurality of medical images;
performing image analysis on the plurality of medical images to extract a plurality of medical images each including a lesion candidate region;
for at least two medical images among the extracted plurality of medical images each including the lesion candidate region, calculating lesion information on the lesion candidate region included in each of the at least two medical images; and
based on the lesion information on each of the at least two medical images from each of which the lesion information has been calculated, controlling display of information indicating presence of the lesion candidate region about each of the plurality of medical images each including the lesion candidate region.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is an overall configuration diagram of an image display system in a first embodiment;

FIG. 2 is a block diagram illustrating a functional configuration of an interpretation terminal shown in FIG. 1;

FIG. 3 is a sequence diagram illustrating a flow from imaging to image display in the first embodiment;

FIG. 4 shows an example of a conventional interpretation screen;

FIG. 5 shows an example of a correspondence table;

FIG. 6 shows an example of an interpretation screen to which the present invention is applied;

FIG. 7 shows an example of the interpretation screen to which the present invention is applied;

FIG. 8 shows an example of the interpretation screen to which the present invention is applied;

FIG. 9 shows a modification example of detection marks;

FIG. 10 is an overall configuration diagram of an image display system in a second embodiment; and

FIG. 11 is a sequence diagram illustrating a flow from imaging to image display in the second embodiment.

### DETAILED DESCRIPTION

Hereinafter, one or more embodiments of the present invention will be described with reference to the drawings. However, the scope of the invention is not limited to the disclosed embodiments or illustrated examples.

### First Embodiment

### [Configuration of Image Display System]

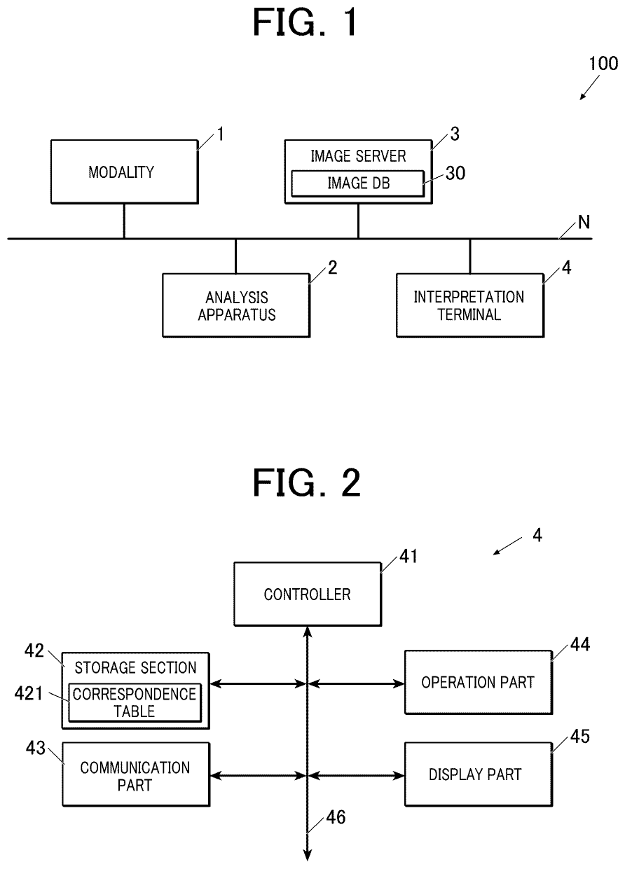

FIG. 1 is a diagram illustrating an example of the system configuration of an image display system 100 (medical image display system) according to a first embodiment.

As illustrated in FIG. 1, the image display system 100 is configured to include a modality 1, an analysis apparatus 2, an image server 3, and an interpretation terminal 4. The apparatuses constituting the image display system 100 are connected to each other via a communication network N such as a local area network (LAN), a wide area network (WAN), or the Internet. Further, each of the apparatuses that constitute the image display system 100 complies with HL7 (Health Level Seven) or DICOM (Digital Image and Communications in Medicine). Communication between the apparatuses is performed in accordance with HL7 or DICOM. Note that the number of modalities 1, the number of analysis apparatuses 2, the number of interpretation terminals 4, and so forth are not particularly limited.

The modality 1 images a subject (an examination site of a patient) and generates a medical image of the subject. In the present embodiment, the modality 1 images a subject and generates a series of medical images including a plurality of medical images. For example, the modality 1 is computed tomography (CT) or magnetic resonance imaging (MRI), and generates a plurality of consecutive medical images (tomographic images) at different slice positions. Alternatively, the modality 1 is a dynamic radiographic imaging apparatus, a fluoroscope, or an ultrasonic diagnostic apparatus, and generates a plurality of temporally consecutive medical images (frame images). In the present embodiment, a case where the modality 1 is CT or MRI will be described as an example.

The modality 1 attaches, to the medical images, image attribute information on the generated medical images, and transmits the medical images to the analysis apparatus 2 and the image server 3.

Here, the image attribute information includes patient information, examination information, image identification information, and the like. The patient information includes a patient ID, a patient name, date of birth, age, sex, height, weight, and the like. The examination information includes an examination ID, an examination date and time, a modality type, an examination site, a requesting department, an examination purpose, and the like. The image identification information includes an instance number, and a unique identifier (UID). The instance number is a number indicating the ordinal position of a certain medical image in the series of medical images. The UID is information for uniquely identifying a medical image.

The analysis apparatus 2 performs image analysis by computer processing on the series of medical images transmitted from the modality 1 to extract medical images each including a lesion candidate region, calculates, for each of the extracted medical images, lesion information on the lesion candidate region, and transmits (outputs) each calculated lesion information to the image server 3.

The analysis apparatus 2 has functions as an acquisition section, an extraction section, and a calculation section of the present invention. The acquisition section acquires, via a communication part (not illustrated), the series of medical images including a plurality of medical images transmitted from the modality 1. The extraction section performs image analysis on a plurality of medical images among the series of medical images transmitted from the modality 1 to extract a plurality of medical images each including a lesion candidate region. The calculation section calculates, for at least two medical images among the plurality of medical images each including a lesion candidate region extracted by the extraction section, lesion information on the lesion candidate region included in each of the medical images.

The analysis apparatus 2 realizes functions as the extraction section and the calculation section by analysis (AI analysis) using, for example, a CAD or a machine learning model. As a method of the machine learning model, for example, a convolutional neural network (CNN) or fully convolutional networks (FCN) can be used, but the method is not particularly limited. The functions of the extraction section and the calculation section are realized, for example, by cooperation between a processor such as a CPU and a program (machine learning model) stored in a storage section (not illustrated).

Here, the lesion information calculated by the analysis apparatus 2 is at least one of the risk degree of a lesion, the certainty factor of the lesion, the size of the lesion, the type of the lesion, the mode of the change in the lesion between the past and the present, and the anatomical structure information on the lesion. The risk degree of lesion is, for example, the risk of the lesion in the lesion candidate region, such as the likelihood of becoming cancerous, expressed as a numerical value or a class. The certainty factor of lesion is a value indicating the probability that the lesion candidate region is a lesion. The size of lesion is a size (for example, a diameter or a radius) of the lesion candidate region. The type of lesion is the type of a lesion corresponding to the lesion candidate region. Examples of the type of lesion in a case where the medical image is a medical image of a chest include a node, a tumor, and pneumothorax. The mode of change in lesion between the past and this time is information indicating the mode of change (increase or decrease in size, new appearance, division, integration, disappearance, or the like) in lesion (lesion candidate region) between the past (previous) examination and this time examination. The anatomical structure information on a lesion is information indicating an anatomical structure (site) in which the lesion (lesion candidate region) exists.

In a case where the lesion information is lesion information on a thoracic node, the lesion information may be information on at least one of the size of the lesion, the difference between the previous and current lesions, the type of lesion (Solid/P-Solid/GGN), and the site of the lesion. The size of the lesion may be the size itself of the lesion candidate region, or may be information indicating whether or not the size of the lesion is equal to or larger than a predetermined threshold. The difference between the past and current lesions is information indicating the difference (value of increase or decrease in size) in the lesion (lesion candidate region) between the past (last time) examination and this time examination. The site of the lesion is information (for example, anatomical structure information) indicating a site where the lesion candidate region is present.

In a case where the lesion information is lesion information on a fracture, the lesion information may be information on at least one of a fracture site and a fracture degree. The fracture site is a more detailed site than the above-described anatomical structure. For example, when the second rib is fractured, the information on the above-described anatomical structure is “rib”, but the fracture site is “second rib”. The fracture degree is information indicating the severity of a fracture.

In a case where the lesion information is lesion information on a head, the lesion information may be information on at least one of the type of lesion (high absorption/low absorption, brain aneurysm, white matter) and the site of the lesion. The site of the lesion is information (for example, anatomical structure information) indicating a site where the lesion candidate region is present.

Note that it is preferable that the lesion information also includes position information (coordinates) of the lesion candidate region in the medical image.

The analysis apparatus 2 associates and transmits, to the image server 3, each calculated lesion information with the image attribute information on the medical image from which the lesion information has been calculated.

The image server 3 is, for example, a server of a picture archiving and communication system (PACS) and is an image management apparatus that stores and manages the medical images output from the modality 1. The image server 3 is an image server of a cloud type, an on-premise type, or a workstation type.

The image server 3 is provided with an image database (DB) 30. The image DB 30 is a database for storing the medical images transmitted from the modality 1 and the lesion information transmitted from the analysis apparatus 2. The image DB 30 includes an image management table that stores information on each of the medical images stored in the image DB 30. The image management table stores, for example, patient information, examination information, image identification information, and lesion information for each medical image in an associated manner.

The interpretation terminal 4 is, for example, a client of PACS (PACS viewer). The interpretation terminal 4 reads medical images from the image server 3 and displays the same for interpretation. The interpretation terminal 4 may be an application-type viewer that operates with an application or a browser-type viewer that operates with a browser. In the present embodiment, a case where the interpretation terminal 4 is an application-type viewer will be described as an example.

FIG. 2 is a block diagram illustrating a functional configuration of the interpretation terminal 4.

As shown in FIG. 2, the interpretation terminal 4 includes a controller 41, a storage section 42, a communication part 43, an operation part 44, a display part 45, and the like, and these parts are connected to each other via a bus 46.

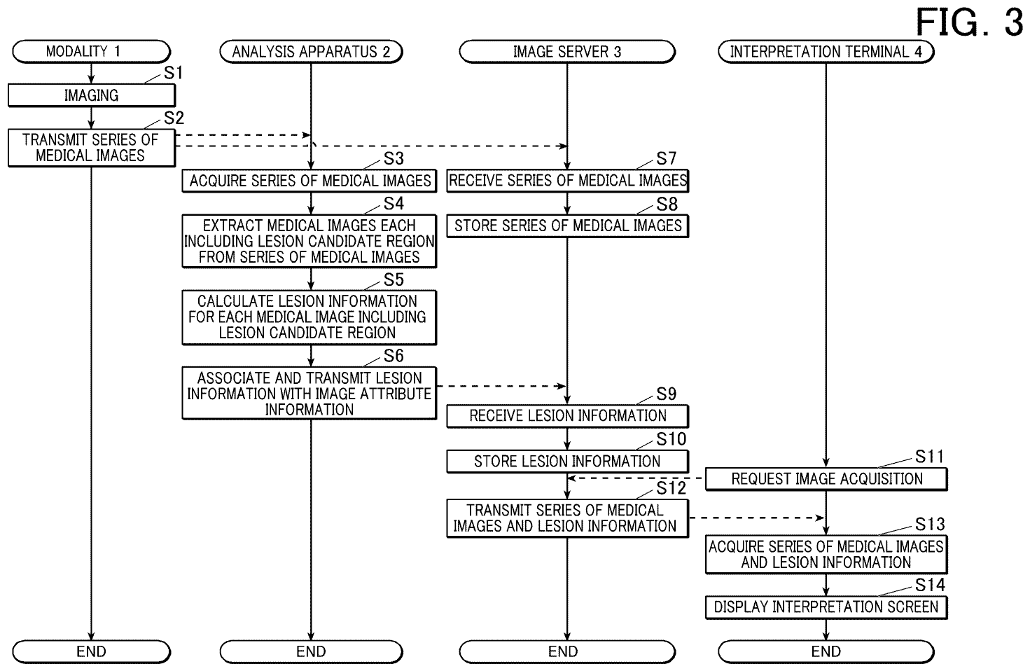

The controller 41 includes a central processing unit (CPU), a random access memory (RAM), and the like. The controller 41 comprehensively controls the operation of each part of the interpretation terminal 4. For example, the CPU of the controller 41 reads various process programs stored in the storage section 42, develops the programs in the RAM, and executes processes on the interpretation terminal 4 side shown in FIG. 3 in accordance with the developed programs. The controller 41 functions as a controller of the present invention.

The storage section 42 includes a hard disk drive (HDD), a semiconductor memory, and the like. The storage section 42 stores a system program(s), application programs for executing various processes, data such as parameters required for executing the programs, and the like.

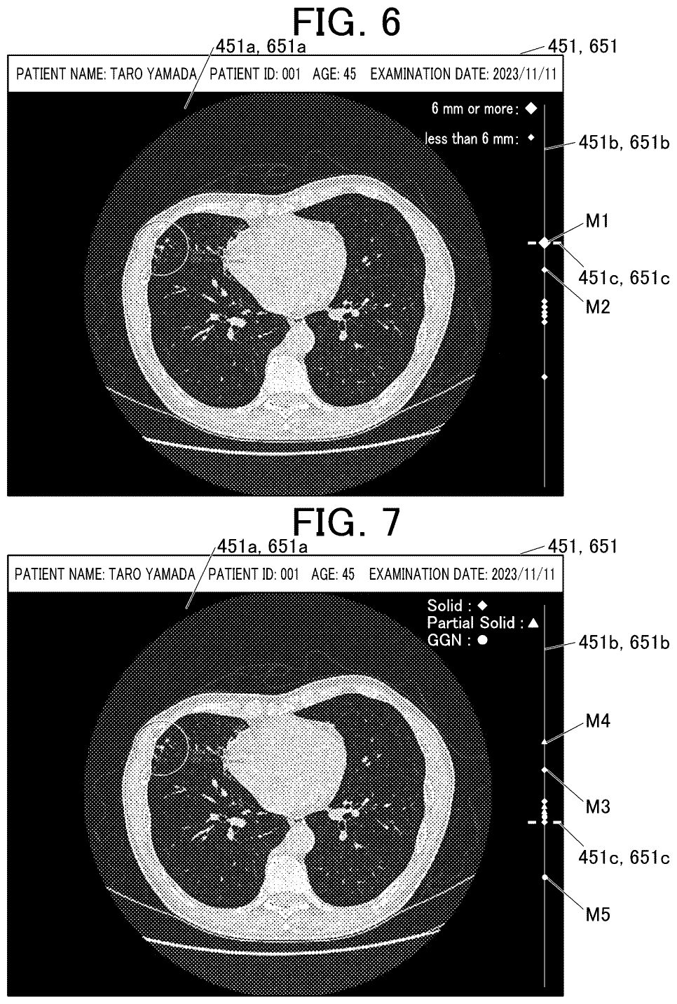

For example, the storage section 42 stores items of lesion information to be used for controlling display forms of detection marks (M1 to M6) in an interpretation screen 451 (see FIGS. 6 to 8). The items of lesion information to be used for controlling the display forms of the detection marks (M1 to M6) can be set by the user operating the operation part 44. A correspondence table 421 (see FIG. 5) is stored in the storage section 42. Details of the correspondence table 421 will be described later.

Further, the storage section 42 has a region for temporarily storing medical images, image attribute information on each of them and lesion information on each of them acquired from the image server 3.

The communication part 43 is configured by a network interface or the like. The communication part 43 transmits and receives data to and from an external apparatus connected via the communication network N.

The operation part 44 includes a keyboard having various keys, a pointing device such as a mouse, or a touch screen superimposed on the display part 45. The operation part 44 outputs, to the controller 41, an operation signal input by a user's key operation on the keyboard, mouse operation, or touch operation on the touch screen.

The display part 45 is composed of a monitor such as a liquid crystal display (LCD). The display part 45 displays various screens according to instructions of display signals input from the controller 41.

### [Operation of Image Display System 100]

Next, the operation of the image display system 100 will be described.

FIG. 3 is a sequence diagram illustrating a flow from imaging to image display in the image display system 100. Hereinafter, a flow from imaging to image display in the image display system 100 will be described with reference to FIG. 3.

First, the modality 1 images a subject and acquires a series of medical images including a plurality of medical images (Step S1).

Next, the modality 1 attaches image attribute information to each of the acquired series of medical images and transmits the same to the analysis apparatus 2 and the image server 3 (Step S2).

The analysis apparatus 2 receives (acquires) the series of medical images transmitted from the modality 1 (Step S3).

Next, the analysis apparatus 2 performs image analysis on a plurality of medical images among the series of medical images transmitted from the modality 1 to extract a plurality of medical images each including a lesion candidate region (Step S4).

Note that when the same lesion candidate region appears across a plurality of medical images, the analysis apparatus 2 extracts a medical image in which the lesion candidate region appears largest as a medical image including the lesion candidate region, for example.

Next, the analysis apparatus 2 calculates, for at least two of the plurality of medical images each including a lesion candidate region, the lesion information on the lesion candidate region included in each of the medical images (Step S5).

Next, the analysis apparatus 2 associates and transmits, to the image server 3, each calculated lesion information with the image attribute information on the medical image from which the lesion information has been calculated (Step S6).

When receiving the series of medical images transmitted from the modality 1 (Step S7), the image server 3 stores the series of medical images in the image DB 30 (Step S8). That is, the image server 3 stores the received series of medical images in the image DB 30, and also writes the image attribute information on each of the series of medical images in the image management table.

Further, when receiving each calculated lesion information transmitted from the analysis apparatus 2 (Step S9), the image server 3 stores the lesion information in the image DB 30 (Step S10). That is, the image server 3 writes each received lesion information, of the image management table, in a record in which the image attribute information on the medical image corresponding to the lesion information is written.

In the interpretation terminal 4, when examination information or the like on medical images to be displayed is specified by the operation part 44, the controller 41 causes the communication part 43 to transmit a request to acquire images in the specified examination to the image server 3 (Step S11).

The image server 3 reads the series of medical images in the specified examination and the (pieces of) lesion information thereon from the image DB 30, and transmits them to the interpretation terminal 4 (Step S12).

In the interpretation terminal 4, when the communication part 43 receives (acquires) the series of medical images transmitted from the image server 3 (Step S13), the controller 41 causes the display part 45 to display the interpretation screen 451 for displaying the received medical images (Step S14).

The user operates the interpretation screen 451 to perform interpretation.

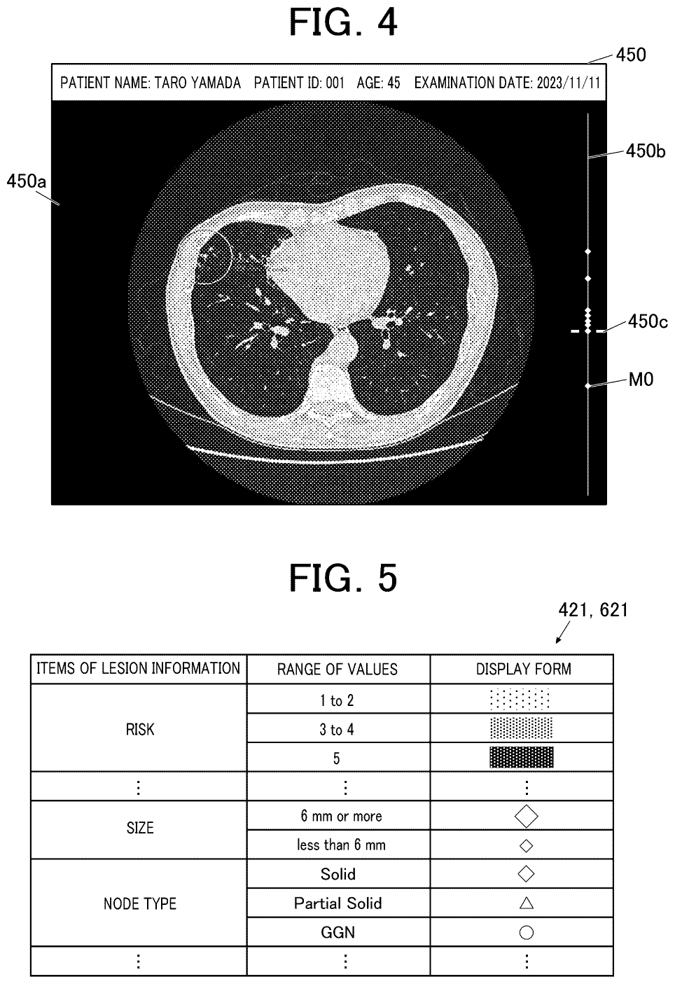

FIG. 4 shows an example of a conventional interpretation screen 450.

As illustrated in FIG. 4, the conventional interpretation screen 450 is provided with a medical image display region 450a and a slider bar 450b having a slider 450c.

The medical image display region 450a is a region in which a medical image corresponding to the position of the slider 450c among a series of medical images to be displayed is displayed.

The slider bar 450b is a bar-shaped UI (User Interface) for specifying a medical image to be displayed among a series of medical images, that is, an operation means. The longitudinal direction of the slider bar 450b indicates a direction perpendicular to the plane (section) of the subject in a series of medical images. One end (e.g., the upper end) of the slider bar 450b corresponds to the slice position of the medical image captured first, and the other end (e.g., the lower end) corresponds to the slice position of the medical image captured last. The position of the slider 450c indicates the slice position of the medical image currently displayed in the medical image display region 450a. When the slider 450c is moved on the slider bar 450b, the medical image displayed in the medical image display region 450a is changed to a medical image corresponding to the position of the slider 450c after the movement.

A detection mark M0 is displayed on the slider bar 450b of the interpretation screen 450. The detection mark M0 is information indicating presence of a lesion candidate region about a medical image including the lesion candidate region. The detection mark M0 is displayed in association with the position of the medical image including the lesion candidate region on the slider bar 450b.

In the interpretation screen 450 shown in FIG. 4, detection marks M0 are displayed in the same display form at all the positions of the medical images each including a lesion candidate region. Therefore, if a large number of medical images each including a lesion candidate region exist and a large number of detection marks M0 are displayed, the user cannot know which medical image is to be preferentially viewed. In Japanese Unexamined Patent Publication No. 2004-173910, display is performed so as to distinguish a display-done object and a dimply-not-done object from one another, but the user cannot recognize a medical image that should be interpreted with priority.

Therefore, based on the lesion information on each medical image from which the lesion information has been calculated, the controller 41 controls display of the information indicating presence of a lesion candidate region about each medical image including a lesion candidate region. As an embodiment, the controller 41 controls, in display of the interpretation screen 451, display of a detection mark as the information indicating presence of a lesion candidate region about a medical image including a lesion candidate region, based on the lesion information on each medical image from which the lesion information has been calculated. A medical image display region 451a, a slider bar 451b, and a slider 451c in the interpretation screen 451 are the same as the medical image display region 450a, the slider bar 450b, and the slider 450c described above, respectively, and thus the description thereof is incorporated herein by reference.

For example, based on the lesion information on each medical image from which the lesion information has been calculated, the controller 41 makes the display form of at least one detection mark different from the display form of the other detection marks, among the detection marks each as the information indicating presence of a lesion candidate region about a medical image including a lesion candidate region. For example, the controller 41 controls, based on the lesion information on each medical image, at least one of the size, the color, and the design of a detection mark(s), thereby making the display form of at least one detection mark different from the display form of the other detection marks.

For example, in Step S14, the controller 41 controls display of detection marks on the basis of the lesion information on each medical image by using the items of the lesion information and the correspondence table 421 that are used for the control of the display forms of the detection marks and stored in the storage section 42.

FIG. 5 shows an example of the correspondence table 421. As illustrated in FIG. 5, the correspondence table 421 stores, for each item of lesion information, ranges of values and display forms of detection marks to be displayed for/at the positions of medical images each including a lesion candidate region having a value that falls in one of the ranges of values, in association with each other. The correspondence table 421 stores display forms different from one another in at least one of the size, the color, and the design of detection mark for each range of values for each item. Note that the values are not limited to numerical values, but include character strings and the like.

The controller 41 acquires, for each medical image from which the lesion information has been calculated, the item(s) of lesion information stored in the storage section 42 and to be used for controlling the display form. Next, the controller 41 refers to the correspondence table 421 and performs control to display a detection mark in a display form corresponding to the range of values in which the value of the item of the acquired lesion information falls, at the position corresponding to the medical image on the slider bar 451b. Thus, the controller 41 can perform control such that at least one of the size, the color, and the design of detection mark to be displayed is different between medical images with different ranges of values for a predetermined item of lesion information. That is, the controller 41 can make the display form of at least one detection mark different from the display form(s) of the other detection mark(s) for medical images having values of a predetermined item of lesion information, the values falling in different ranges of values for.

FIG. 6 shows an example of the interpretation screen 451. FIG. 6 illustrates an example of display of the interpretation screen 451 in a case where the item of the lesion information to be used for controlling the display forms of the detection marks is “Size”, and a rhombus of a first size is associated with “6 mm or more”, and a rhombus of a second size smaller than the first size is associated with “less than 6 mm”, in the correspondence table 421.

As illustrated in FIG. 6, in the interpretation screen 451, detection marks M1 and M2 are displayed on the slider bar 451b. The detection marks M1 and M2 are the information indicating presence of a lesion candidate region about a medical image including a lesion candidate region. The detection mark M1 is displayed on the slider bar 451b in association with the position of a medical image including a lesion candidate region having a size of 6 mm or more. The detection mark M2 is displayed on the slider bar 451b in association with the position of a medical image including a lesion candidate region having a size of less than 6 mm.

As described above, in the interpretation screen 451 illustrated in FIG. 6, the detection mark M1 is displayed for the position of a medical image including a lesion candidate region of 6 mm or more, and the detection mark M2 different from the detection mark M1 in size is displayed for the position of a medical image including a lesion candidate region of less than 6 mm. Since the user can recognize which medical image includes a lesion candidate region having a size of 6 mm or more, the user can easily recognize which medical image should be given priority for interpretation.

FIG. 7 shows another example of the interpretation screen 451. FIG. 7 illustrates an example of display of the interpretation screen 451 in a case where the item of the lesion information to be used for controlling the display forms of the detection marks is “Node Type”, and a rhombus is associated with “Solid”, a triangle is associated with “Partial Solid”, and a circle is associated with “GGN”, in the correspondence table 421.

As shown in FIG. 7, in the interpretation screen 451, detection marks M3 to M5 are displayed on the slider bar 451b. The detection marks M3 to M5 are the information indicating presence of a lesion candidate region about a medical image including a lesion candidate region. The detection mark M3 is displayed on the slider bar 451b in association with the position of a medical image including a lesion candidate region having a node type of “Solid”. The detection mark M4 is displayed on the slider bar 451b in association with the position of a medical image including a lesion candidate region having a node type of “Partial Solid”. The detection mark M5 is displayed on the slider bar 451b in association with the position of a medical image including a lesion candidate region having a node type of “GGN”.

As described above, in the interpretation screen 451 shown in FIG. 7, the detection mark M3 displayed for the position of a medical image including a lesion candidate region having a node type of “Solid”, the detection mark M4 displayed for the position of a medical image including a lesion candidate region having a node type of “Partial Solid”, and the detection mark M5 displayed for the position of a medical image including a lesion candidate region having a node type of “GGN” are displayed with different designs. Since the user can recognize which medical image includes which type of node, the user can easily recognize which medical image should be given priority for interpretation.

Further, the controller 41 may control display of the information indicating presence of a lesion candidate region about each of a plurality of medical images each including a lesion candidate region by performing control not to display (to hide) at least one of the pieces of the information indicating presence of a lesion candidate region, on the basis of the lesion information on each medical image from which the lesion information has been calculated.

For example, in the correspondence table 421, for each item of lesion information, a detection mark having a predetermined shape, size, and color is stored in association with the range of values to be preferentially interpreted, and “No-display” is stored in association with the other ranges of values. In Step S14, the controller 41 acquires, for each medical image from which the lesion information has been calculated, the item(s) of lesion information stored in the storage section 42 and to be used for controlling the display form of the detection mark. Next, the controller 41 refers to the correspondence table 421, and if the display form corresponding to the range of values of the item of the acquired lesion information is a predetermined detection mark, the controller 41 performs control to display the detection mark at the position corresponding to the medical image on the slider bar 451b. If the display form corresponding to the range of values of the item of the acquired lesion information is “No-display”, no detection mark is displayed at the position corresponding to the medical image on the slider bar 451b.

In this way, on the slider bar 451b, the detection mark can be displayed for the position of the medical image whose lesion information satisfies a predetermined condition, and no detection mark can be displayed for the position of the medical image whose lesion information does not satisfy the predetermined condition, among the medical images each including a lesion candidate region. Therefore, the user can easily recognize which medical image should be given priority for interpretation.

In Step S14, the controller 41 may control display of the detection mark, which is the information indicating presence of a lesion candidate region about a medical image including a lesion candidate region, on the basis of a comparison between pieces of lesion information on medical images for each of which the lesion information has been calculated.

For example, in a case where a plurality of medical images for each of which lesion information has been calculated include lesion information indicating that the size of a lesion is equal to or larger than a diameter of 2 mm, the controller 41 performs control to display the detection marks M6 at positions corresponding to medical images having pieces of the lesion information indicating the size being the top four. In addition, the controller 41 performs control not to display detection marks corresponding to medical images other than the above.

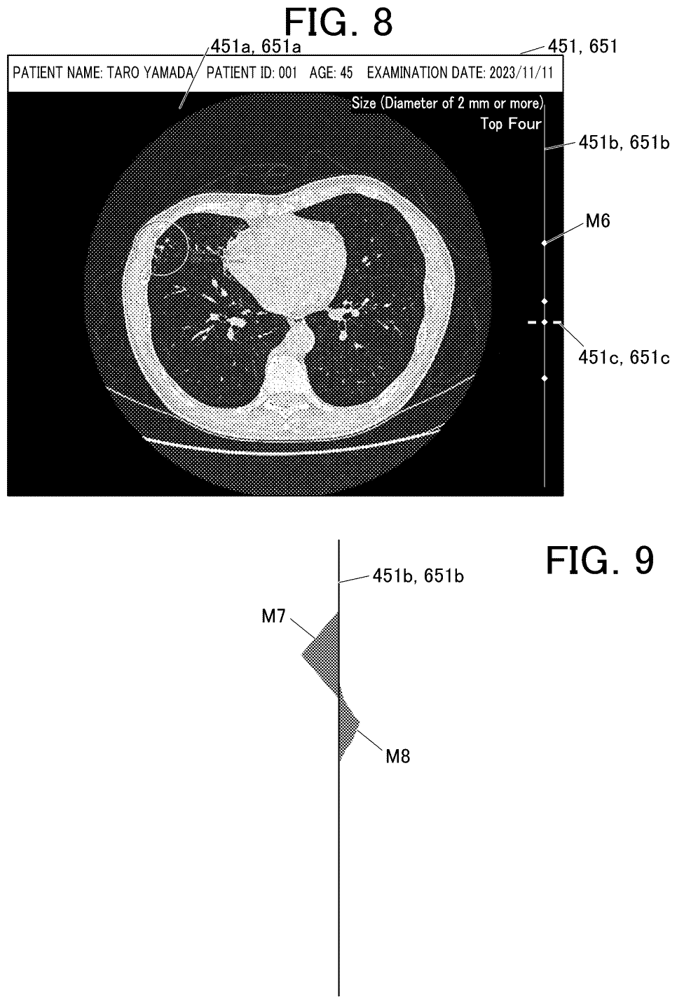

FIG. 8 shows another example of the interpretation screen 451. FIG. 8 illustrates an example in which the detection marks M6 are displayed at the positions corresponding to the medical images whose lesion information indicates that the size of the lesion candidate region is a diameter of 2 mm or more and in the top four, and no detection marks are displayed at the positions corresponding to the other medical images.

As described above, in the interpretation screen 451 shown in FIG. 8, the detection marks M6 are displayed only at the positions corresponding to the medical images whose lesion information indicates that the size of the lesion candidate region is 2 mm or more and in the top four. Therefore, the user can easily recognize which medical image should be given priority for interpretation.

When controlling display of the detection mark, which is the information indicating presence of a lesion candidate region about a medical image including a lesion candidate region, on the basis of the comparison between pieces of lesion information on the medical images for each of which the lesion information has been calculated, the controller 41 does not need to compare all the lesion candidate regions of the plurality of medical images. In addition, it is unnecessary to control display of all pieces of information indicating presence of a lesion candidate region. For example, the controller 41 may control, on the basis of at least first lesion information on a first lesion candidate region included in a first medical image and second lesion information on a second lesion candidate region included in a second medical image, display of at least the information indicating presence of a lesion candidate region about the first medical image.

### Modification Example 1

In the first embodiment described above, when the same lesion candidate region appears across a plurality of medical images, the analysis apparatus 2 extracts the medical image in which the lesion candidate region appears largest as the medical image including the lesion candidate region. Alternatively, when the same lesion candidate region appears across a plurality of medical images, the analysis apparatus 2 may extract all the medical images in which the lesion candidate region appears as the medical images including the lesion candidate region, and calculate the lesion information on the extracted medical images. Then, the controller 41 of the interpretation terminal 4 may control, on the basis of the lesion information on each medical image from which the lesion information has been calculated, display of the information indicating presence of a lesion candidate region about each of the plurality of medical images including the lesion candidate region. For example, when two lesion candidate regions are detected across a plurality of medical images, the controller 41 may perform control to display information M7 indicating presence of one lesion candidate region on the left side of the slider bar 451b and information M8 indicating presence of the other lesion candidate region on the right side of the slider bar 451b, as shown in FIG. 9. At the time, the controller 41 performs control to display the information M7 and the information M8 with the length in a direction (left or right) orthogonal to the slider bar 451b as the size (radius or diameter) of the lesion candidate region detected from each medical image and the up-down direction as the position of the medical image. Thus, the user can recognize the areas and the sizes of two lesion candidate regions included in a series of medical images.

### Modification Example 2

In the image display system 100 of the first embodiment, the case where the analysis apparatus 2 has functions as the acquisition section, the extraction section, and the calculation section, and the interpretation terminal 4 has functions as the controller and the display part has been described as an example. However, the apparatuses having the functions as the acquisition section, the extraction section, the calculation section, the controller, and the display part are not limited to the above-described example.

For example, if the image display system 100 is not provided with the analysis apparatus 2, the interpretation terminal 4 may be configured as a medical image display apparatus of the present invention to have all the functions of the acquisition section, the extraction section, the calculation section, the controller, and the display part. For example, the storage section 42 of the interpretation terminal 4 stores a program for causing the controller 41 to execute the functions as the acquisition section, the extraction section, the calculation section, and the controller of the present invention. The controller 41 executes the functions of the acquisition section, the extraction section, the calculation section, and the controller in cooperation with the program stored in the storage section 42, and causes the display part 45 to function as the display part.

If the image display system 100 is not provided with the analysis apparatus 2, the image server 3 may be configured as the medical image display apparatus of the present invention to have the functions of the acquisition section, the extraction section, the calculation section, and the controller. For example, the image server 3 executes the functions of the acquisition section, the extraction section, the calculation section, and the controller in cooperation with a processor such as a CPU and a program stored in a storage section. Note that when controlling display of the information indicating presence of a lesion candidate region about each of a plurality of medical images, the image server 3 may, as the controller, perform control to cause a display part included in the image server 3 to display the information. Alternatively, when controlling display of the information indicating presence of a lesion candidate region about each of a plurality of medical images, the image server 3 may control, as the controller, the display part of the interpretation terminal 4 to display the information.

Further, the analysis apparatus 2 may be configured to include all the functions of the acquisition section, the extraction section, the calculation section, the controller, and the display part as the medical image display apparatus of the present invention. For example, the analysis apparatus 2 may include a display part, and when controlling display of the information indicating presence of a lesion candidate region about each of a plurality of medical images, the analysis apparatus 2 may control, as the controller, the display part included in the analysis apparatus 2 to display the information. Alternatively, when controlling display of the information indicating presence of a lesion candidate region about each of a plurality of medical images, the analysis apparatus 2 may control, as the controller, the display part of the interpretation terminal 4 to display the information.

### Second Embodiment

Hereinafter, a second embodiment of the present invention will be described.

The image display system 100 illustrated in FIG. 1 is provided in a relatively large-scale medical facility. On the other hand, in a small-scale medical facility such as a medical facility owned by a medical practitioner or a clinic, a medical image display apparatus integrated with functions of an analysis apparatus, an image server, and an interpretation terminal is often used. Hereinafter, an example in which the present invention is applied to such a medical image display apparatus for small-scale medical facilities will be described.

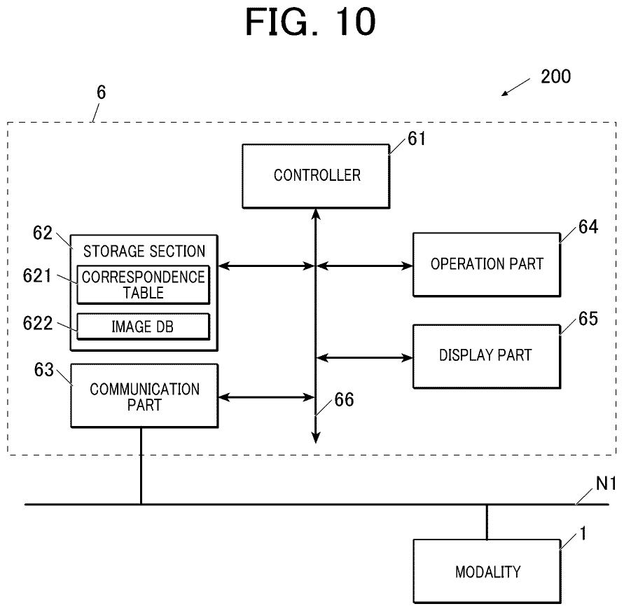

FIG. 10 shows an example of an image display system 200 in a second embodiment. As illustrated in FIG. 10, the image display system 200 includes a modality 1 and a medical image display apparatus 6. The modality 1 and the medical image display apparatus 6 are connected to each other via a communication network N1 such as a LAN. The modality 1 is the same as that described in the first embodiment, and thus the description is incorporated herein by reference.

As shown in FIG. 10, the medical image display apparatus 6 includes a controller 61, a storage section 62, a communication part 63, an operation part 64, and a display part 65 that are connected to each other via a bus 66.

The controller 61 includes a CPU, a RAM, and the like. The controller 61 comprehensively controls the operation of each part of the medical image display apparatus 6. Specifically, the CPU of the controller 61 reads various process programs stored in the storage section 62, develops the programs in the RAM, and executes various processes in accordance with the developed programs.

The storage section 62 includes an HDD, a semiconductor memory and/or the like. The storage section 62 stores a system program(s), application programs for executing various processes, data such as parameters necessary for execution of the programs, and the like. The programs stored in the storage section 62 include a program for causing a computer (controller 61) to realize the functions as the acquisition section, the extraction section, the calculation section, and the controller of the present invention. The acquisition section acquires, via a communication part (not illustrated), the series of medical images including a plurality of medical images transmitted from the modality 1. The extraction section performs image analysis on a plurality of medical images among the series of medical images transmitted from the modality 1 to extract a plurality of medical images each including a lesion candidate region. The calculation section calculates, for at least two medical images among the plurality of medical images including the lesion candidate region extracted by the extraction section, lesion information on the lesion candidate region included in each of the medical images. The controller controls display of the information indicating presence of a lesion candidate region about each of the plurality of medical images including a lesion candidate region, on the basis of the lesion information on each medical image whose lesion information has been calculated. The lesion information is the same as that described in the first embodiment, and thus, the description is incorporated herein by reference.

The controller 61 realizes the functions as the extraction section and the calculation section by analysis (AI analysis) using, for example, a CAD or a machine learning model in cooperation with the program stored in the storage section 62. As a method of the machine learning model, for example, CNN or FCN can be used, but the method is not particularly limited.

The storage section 62 includes a correspondence table 621 and an image DB 622. The correspondence table 621 is the same as the correspondence table 421 described in the first embodiment, and therefore the description thereof is incorporated herein by reference. The image DB 622 is a database for storing the medical images transmitted from the modality 1 and the lesion information calculated from each of the medical images. The image DB 622 includes an image management table that stores information on the medical images stored in the image DB 622. The image management table stores, for example, patient information, examination information, image identification information, and lesion information for each medical image in an associated manner.

The communication part 63 includes a network interface and/or the like. The communication part 63 transmit and receive data to and from an external apparatus connected via the communication network N1.

The operation part 64 includes a keyboard having various keys, a pointing device such as a mouse, or a touch screen superimposed on the display part 65. The operation part 64 outputs, to the controller 61, an operation signal input by a user's key operation on the keyboard, mouse operation, or touch operation on the touch screen.

The display part 65 is composed of a monitor such as an LCD. The display part 65 displays various screens according to instructions of display signals input from the controller 61.

Next, the operation of the image display system 200 of the second embodiment will be described.

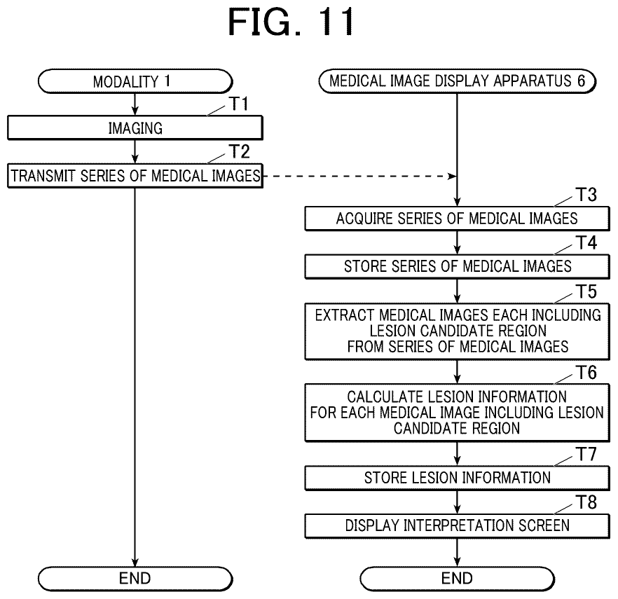

FIG. 11 is a sequence diagram showing a flow from imaging to image display in the image display system 200. Hereinafter, a flow from imaging to image display in the image display system 200 will be described with reference to FIG. 11.

First, the modality 1 images a subject and acquires a series of medical images including a plurality of medical images (Step T1).

Next, the modality 1 attaches image attribute information to each of the acquired series of medical images and transmits the same to the medical image display apparatus 6 (Step T2).

The controller 61 of the medical image display apparatus 6 receives (acquires) the series of medical images transmitted from the modality 1 with the communication part 63 (Step T3), and stores the acquired series of medical images in the image DB 622 (Step T4). That is, the controller 61 stores the received series of medical images in the image DB 622, and also writes the image attribute information on each of the series of medical images in the image management table.

Next, the controller 61 performs image analysis on a plurality of medical images among the series of medical images transmitted from the modality 1 to extract a plurality of medical images each including a lesion candidate region (Step T5).

The process in Step T5 is the same as the process in Step S4 in FIG. 3, and thus the description thereof is incorporated herein by reference.

Next, the controller 61 calculates, for at least two of the plurality of medical images each including a lesion candidate region, the lesion information on the lesion candidate region included in each of the medical images (Step T6).

Next, the controller 61 associates and stores, in the image DB 622, each calculated lesion information with the medical image from which the lesion information has been calculated (Step T7).

The controller 61 writes each lesion information, of the image management table of the image DB 622, in a record in which the image attribute information on the medical image corresponding to the lesion information is written.

Then, the controller 61 causes the display part 65 to display an interpretation screen 651 for displaying the medical images received from the modality 1 (Step T8).

In the interpretation screen 651 displayed under the control of the controller 61 in Step T8, as shown in FIG. 6 to FIG. 8, a medical image display region 651a, a slider bar 651b, and a slider 651c are displayed. The medical image display region 651a, the slider bar 651b, and the slider 651c are the same as the medical image display region 451a, the slider bar 451b, and the slider 451c, respectively, and thus the description thereof is incorporated herein by reference.

On the slider bar 651b of the interpretation screen 651, detection marks (e.g., M1 and M2 in FIGS. 6, M3 to M5 in FIG. 7, M6 in FIG. 8, etc.) are displayed. The detection marks are the information indicating presence of a lesion candidate region about a medical image including a lesion candidate region. A detection mark is displayed on the slider bar 651b in association with the position of a medical image including a lesion candidate region.

Based on the lesion information on each medical image from which the lesion information has been calculated, the controller 61 makes the display form of at least one detection mark different from the display form of the other detection marks, among the detection marks each as the information indicating presence of a lesion candidate region about a medical image including a lesion candidate region. The method for controlling the display forms of the detection marks by the controller 61 is the same as the method for controlling the display forms of the detection marks by the controller 41 described in the first embodiment and the modification examples thereof, and thus the description thereof is incorporated herein by reference.

The user operates the slider 651b of the slider bar 651c of the interpretation screen 651 to display a desired medical image in the medical image display region 651a for interpretation. On the slider bar 651b, a detection mark(s), which is the information indicating presence of a lesion candidate region about a medical image including a lesion candidate region, is displayed in the display form corresponding to the lesion information on a medical image. Therefore, the user can easily recognize which medical image should be given priority for interpretation.

As described above, the image display system 100 and the medical image display apparatus 6 each include the acquisition section (hardware processor) that acquires a series of medical images including a plurality of medical images; the extraction section (hardware processor) that performs image analysis on the plurality of medical images to extract a plurality of medical images each including a lesion candidate region; the calculation section (hardware processor) that for at least two medical images among the extracted plurality of medical images each including the lesion candidate region, calculates lesion information on the lesion candidate region included in each of the at least two medical images; and the controller (hardware processor) that based on the lesion information on each of the at least two medical images from each of which the lesion information has been calculated, controls display of information indicating presence of the lesion candidate region about each of the plurality of medical images each including the lesion candidate region.

Therefore, it is possible to display, based on the lesion information, which medical image should be preferentially viewed among a plurality of medical images each including a lesion candidate region in an easily understandable manner.

Note that the present invention is not limited to the above-described embodiments, and various modifications can be made without departing from the scope of the present invention.

For example, in the above embodiments, the case has been described as an example in which the number of items of lesion information used to control the display form(s) of the information (detection mark(s) or the like) indicating presence of a lesion candidate region about a medical image including a lesion candidate region is one. However, the display form of the information may be controlled based on a plurality of items of lesion information. For example, the controller 41 (61) scores each of the plurality of items in the lesion information according to the range of values, multiplies the score of each item by a weight coefficient predetermined for each item, and sums the results. Then, based on a table in which a correspondence relationship between the range of summed values and the display form of the above information is stored, the controller 41 (61) performs control to display the information in the display form corresponding to the summed value. Thus, the information indicating presence of a lesion candidate region about a medical image including a lesion candidate region (detection marks, etc.) can be displayed in a display manner reflecting a plurality of items of lesion information.

Further, although in the above embodiments, the case where a series of medical images in the present invention is a plurality of consecutive medical images (tomographic images) at different slice positions generated by CT or MRI has been described as an example, this is not a limitation. For example, the series of medical images may be a plurality of temporally consecutive medical images, such as dynamic radiographic images, fluoroscopic images, or ultrasonic images. In this case, the longitudinal direction of the slider bar 451b (651b) indicates the time axis. The position of the slider 451c (651c) indicates the time position of the medical image currently displayed in the medical image display region 451a (651a).

Note that in the above embodiments, the case where the longitudinal direction of the slider bar 451b (651b) is the up-down direction has been described as an example, but the longitudinal direction may be the right-left direction. Further, although in the above embodiments, the slider bar 450b (651b) is the operation means for specifying a medical image to be displayed among a series of medical images, the operation means may be an operation means for continuously displaying a series of medical images.

Further, although in the above embodiments, the display form of the detection mark is switched according to the item of the lesion information, this is not a limitation. For example, the interpretation screen 451 (651) may be provided with a switching button, and the controller 41 may switch the display form of the detection mark in response to an operation on the switching button.

Further, in the above embodiments, the lesion candidate regions are detected by image analysis, but regions specified by a radiologist or the like (user) on medical images may be detected as the lesion candidate regions. In this case, the lesion information on each lesion candidate region may be calculated by computer processing or may be input by the radiologist or the like.

Further, in the above description, a hard disk, a nonvolatile semiconductor memory or the like is used as a computer-readable medium storing the program(s) of the present invention, but this is not a limitation. As the computer-readable medium, a portable recording medium such as a CD-ROM is applicable. Further, a carrier wave is also applicable as a medium for providing data of the program(s) of the present invention via a communication line.

The detailed configuration/components and the detailed operation of each apparatus constituting the image display system can also be appropriately modified without departing from the scope of the invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-210229, filed on Dec. 13, 2023, including description, claims, drawings and abstract is incorporated herein by reference.

What is claimed is:

1. A medical image display apparatus comprising a hardware processor that
acquires a series of medical images including a plurality of medical images,
performs image analysis on the plurality of medical images to extract a plurality of medical images each including a lesion candidate region,
for at least two medical images among the extracted plurality of medical images each including the lesion candidate region, calculates lesion information on the lesion candidate region included in each of the at least two medical images, and
based on the lesion information on each of the at least two medical images from each of which the lesion information has been calculated, controls display of information indicating presence of the lesion candidate region about each of the plurality of medical images each including the lesion candidate region.

2. The medical image display apparatus according to claim 1, further comprising:
an operation part to specify a medical image to be displayed among the series of medical images; and
a display that displays a detection mark as the information indicating the presence.

3. The medical image display apparatus according to claim 1,
wherein the plurality of medical images each including the lesion candidate region includes at least a first medical image including a first lesion candidate region and a second medical image including a second lesion candidate region, and
wherein based on at least first lesion information on the first lesion candidate region and second lesion information on the second lesion candidate region, the hardware processor controls display of at least information indicating presence of the first lesion candidate region about the first medical image.

4. The medical image display apparatus according to claim 1, wherein based on the lesion information on each of the at least two medical images from each of which the lesion information has been calculated, the hardware processor controls display of the information indicating the presence of the lesion candidate region about each of the plurality of medical images each including the lesion candidate region.

5. The medical image display apparatus according to claim 1, wherein based on the lesion information on each of the at least two medical images from each of which the lesion information has been calculated, the hardware processor controls display of the information indicating the presence of the lesion candidate region about each of the plurality of medical images each including the lesion candidate region by making a display form of at least one piece of the information indicating the presence different from a display form of another piece of the information indicating the presence, among pieces of the information indicating the presence of the lesion candidate region about the plurality of medical images each including the lesion candidate region.

6. The medical image display apparatus according to claim 1, wherein based on the lesion information on each of the at least two medical images from each of which the lesion information has been calculated, the hardware processor controls display of the information indicating the presence of the lesion candidate region about each of the plurality of medical images each including the lesion candidate region by not displaying at least one piece among pieces of the information indicating the presence of the lesion candidate region about the plurality of medical images each including the lesion candidate region.

7. The medical image display apparatus according to claim 5, wherein the hardware processor makes the display form of the at least one piece of the information indicating the presence of the lesion candidate region and the display form of the another piece thereof by controlling at least one of a size, a color and a design of a mark indicating a position of the lesion candidate region.

8. The medical image display apparatus according to claim 1, wherein the lesion information is information on at least one of a risk degree of a lesion, a certainty factor of the lesion, a size of the lesion, a type of the lesion, a mode of a change in the lesion between past and present, and anatomical structure information on the lesion, calculated from the lesion candidate region.

9. The medical image display apparatus according to claim 1, wherein the lesion information is lesion information on a thoracic node, and is information on at least one of a size of a lesion, a difference in the lesion between past and present, a type of the lesion, and a site of the lesion.

10. The medical image display apparatus according to claim 1, wherein the lesion information is lesion information on a fracture, and is information on at least one of a site of the fracture and a degree of the fracture.

11. The medical image display apparatus according to claim 1, wherein the lesion information is lesion information on a head, and is information on at least one of a type of a lesion and a site of the lesion.

12. The medical image display apparatus according to claim 1, wherein the series of medical images is a plurality of consecutive tomographic images at different slice positions.

13. The medical image display apparatus according to claim 1, wherein the series of medical images is a plurality of temporally consecutive medical images.

14. A medical image display system comprising a hardware processor that
acquires a series of medical images including a plurality of medical images,
performs image analysis on the plurality of medical images to extract a plurality of medical images each including a lesion candidate region,
for at least two medical images among the extracted plurality of medical images each including the lesion candidate region, calculates lesion information on the lesion candidate region included in each of the at least two medical images, and
based on the lesion information on each of the at least two medical images from each of which the lesion information has been calculated, controls display of information indicating presence of the lesion candidate region about each of the plurality of medical images each including the lesion candidate region.

15. A non-transitory computer-readable storage medium storing a program causing a computer to perform:
acquiring a series of medical images including a plurality of medical images;
performing image analysis on the plurality of medical images to extract a plurality of medical images each including a lesion candidate region;
for at least two medical images among the extracted plurality of medical images each including the lesion candidate region, calculating lesion information on the lesion candidate region included in each of the at least two medical images; and
based on the lesion information on each of the at least two medical images from each of which the lesion information has been calculated, controlling display of information indicating presence of the lesion candidate region about each of the plurality of medical images each including the lesion candidate region.

16. A medical image display method comprising:
acquiring a series of medical images including a plurality of medical images;
performing image analysis on the plurality of medical images to extract a plurality of medical images each including a lesion candidate region;
for at least two medical images among the extracted plurality of medical images each including the lesion candidate region, calculating lesion information on the lesion candidate region included in each of the at least two medical images; and
based on the lesion information on each of the at least two medical images from each of which the lesion information has been calculated, controlling display of information indicating presence of the lesion candidate region about each of the plurality of medical images each including the lesion candidate region.
